(19) United States
(12) Patent Application Publication
(10) Pub. No.: US 2007/0031519 A1
(43) Pub. Date: Feb. 8, 2007

(54) Antibacterial bioactive fraction of cinnamon fruit

(75) Inventors: Krishnareddy Guddadarangavvnahally, Karnataka (IN); Pradeep Singh Negi, Karnataka (IN); Lingamullu Jagan Mohan Rao, Karnataka (IN); Kunnumpurath Kurian Sakariah, Karnataka (IN)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: Krishareddy Guddadarangavvanahally et al

(21) Appl. No.: 10/566,338

(22) PCT Filed: Dec. 24, 2003
(86) PCT No.: PCT/IB03/06197

§ 371 Date: May 15, 2006

(51) Int. Cl. A61K 36/54

(52) U.S. Cl. 424/739

(57) ABSTRACT

This invention relates to a composition comprising a bioactive fraction obtained from fruits of Cinnamomum zeylanicum having moisture 4-6%, Greenish white colour and mild salty flavor optionally along with one or more pharmaceutically acceptable additives, use of the bioactive thus obtained as an antibacterial agent and a process for preparing the bioactive fraction.

### CROSS-REFERENCE TO RELATED APPLICATIONS

The present invention claims priority to International Application No. PCT/IB2003/006197 filed Dec. 24, 2003, the teachings of which are incorporated herein by reference.

### FIELD OF THE INVENTION

The present invention relates to a composition comprising a bioactive fraction obtained from the fruits of Cinnamomum zeylanicum, use of the bioactive fraction thus obtained as antibacterial agent and a process for preparing the same from un-conventional parts of Cinnamomum zeylanicum.

### BACKGROUND OF THE INVENTION

The problem of food spoilage has plagued man since ancient times. Microbial activity is a primary mode of deterioration of many foods, and the food industry relies heavily upon use of antimicrobial agents to extend shelf life of food products. With growing concern over the presence of chemical residues in foods, the demand for non-toxic natural preservatives has been rising. Natural antibacterial compounds can be used to replace the chemical additives as they have significant antimicrobial activity (Smid E J, Gorris L G M, Natural antimicrobials for food preservation, In-Shafiurr Rahman M (ed) Handbook of food preservation. Marcel Dekker, Inc, New York, pp. 285-308, 1999).

Cinnamomum zeylanicum Blume (synonym Cinnamomum verum J. S. Presl), the cinnamon of commerce provides various types of oils depending on the part of the plant distilled. Cinnamon is a native of Sri Lanka and tropical Asia. The tree is found in South India up to altitudes of 500 meters but is more common at lower altitudes (The Wealth of India. A Dictonary of Indian Raw Material and Industrial Products. Publication and Information Directorate, New Delhi, 1992, p. 582). A total of 53 compounds of cinnamon leaf oil were identified with the major component being eugenol (about 81-84.5%) (Mallavarapu, G. R., Ramesh, S., Chandrasekhara, R. S., Rajeswara Rao, B. R., Kaul, P. N. and Battacharya, A. K. Flavour and Fragr. J., 10, 239, 1995). The essential oil is reported to have antimicrobial (Dubey, N. K. and Mishra, A. K. Indian Drugs 27, 529-531, 1990), fungitoxic (Saksena, N. K. and Saksena, S. Indian Perfumer, 28, 42-45, 1984), nematicidal (Tiwari, R. Dixit, R. and Dixit, S. N. Indian Perfumer, 38, 98-104, 1994), and leech repelling activities (Nakamura, N. Kiuchi, F., Tsuda, Y., Kondo, K. and Sato, T. Shoyakugaka Zasshi, 44, 183-195, 1990). Cinnamaldehyde (about 75%) and camphor (about 56%) have been reported to be the major components of volatile oils from stem bark and root bark respectively, (Senanayake, W. M., Lee, T. H., and wills, R. B. H., J. Agric. Food. Chem. 26, 822, 1978). Eugenol and cinnamaldehyde are used as antimicrobial agents in pharmaceuticals and oral care product (The Wealth of India. A Dictonary of Indian Raw Material and Industrial Products. Publication and Information Directorate, New Delhi, 1992, p. 582).

Literature survey revealed that, there is no report on the isolation of antibacterial fraction from the fruits of Cinnamomum zeylanicum.

The present invention provides a one step process for preparation of a bioactive fraction, which has antibacterial activity. Hence, the inventors have developed a process for the preparation of bioactive fraction from the unconventional parts of Cinnamomum zeylanicum, which have no commercial value at present.

The principle of the present invention is to provide technology for the preparation of an antibacterial fraction from the unconventional parts of Cinnamomum zeylanicum, which can be used as potential natural preservative.

This invention is related to an efficient process for the large-scale preparation of antibacterial fraction from the unconventional parts of Cinnamomum zeylanicum.

### SUMMARY OF THE INVENTION

According to the present invention, there is provided a composition comprising a bioactive fraction obtained from fruits of Cinnamomum zeylanicum having moisture 4-6%, Greenish white colour and mild salty flavor optionally along with one or more pharmaceutically acceptable additives.

The present invention also provides use of the bioactive thus obtained as an antibacterial agent.

The present invention further provides a process for preparing the bioactive fraction.

The process is efficient and it involves the use of simple extraction methods and solvents, which can be re-used.

### BRIEF DESCRIPTION OF THE DRAWINGS

FIG. 1 is a flow diagram of an embodiment of a process according to the invention.

### DETAILED DESCRIPTION OF THE INVENTION

Accordingly, the present invention relates to a composition comprising a bioactive fraction obtained from fruits of Cinnamomum zeylanicum having

| | |
|---|---|
| Moisture: | 4-6% |
| Color: | Greenish white |
| Flavor: | Mild salty flavor |

optionally along with one or more pharmaceutically acceptable additives.

In an embodiment of the present invention the bioactive fraction is a hexane extract obtained from the fruits of Cinnamomum zeylanicum.

In another embodiment of the present invention the composition has antibacterial activity against gram positive and gram negative bacterial in the range of 200-500 ppm.

In yet another embodiment of the present invention the composition has antibacterial activity against Bacillus cereus, Bacillus subtilis, Bacillus coagulans, Pseudomonas aeruginosa, Staphylococcus aureus.

The present invention provides a novel use of a bioactive fraction obtained from fruits of Cinnamomum zeylanicum having

| | |
|---|---|
| Moisture: | 4-6% |
| Color: | Greenish white |
| Flavor: | Mild salty flavor |

as an antibacterial agent.

In an embodiment of the present invention the bioactive fraction is a hexane extract obtained from the fruits of Cinnamomum zeylanicum.

In another embodiment of the present invention, the bioactive fraction has antibacterial activity against gram positive and gram negative bacterial in the range of 200-500 ppm.

In yet another embodiment of the present invention, the bioactive fraction has antibacterial activity against Bacillus cereus, Bacillus subtilis, Bacillus coagulans, Pseudomonas aeruginosa, Staphylococcus aureus.

The present invention also provides a process for preparing antibacterial bioactive fraction having

| | |
|---|---|
| Moisture: | 4-6% |
| Color: | Greenish white |
| Flavor: | Mild salty flavor |

from the unconventional parts of Cinnamomum zeylanicum, said process comprising the steps of:
(a) extracting the powdered fruits of Cinnamomum zeylanicum with an organic solvent at a temperature in the range of 55-60° C. for a time period in the range of 60-80 mesh.
(b) filtering and concentrating the solvent obtained in step (a) to obtain a concentrate and to recover up to 90% of the solvent;
(c) drying the concentrate obtained in step (b) in a vacuum oven at 40-50° C. under vacuum at 10-25 mm of mercury to obtain the antibacterial bioactive fraction.

In an embodiment of the present invention, the organic solvent used is hexane.

In another embodiment of the present invention, the yield of hexane extract is about 1.5 to 3.0%.

In yet another embodiment of the present invention, the filtration is carried out by conventional methods.

In still another embodiment of the present invention, the concentration temperature is of 55-60° C.

In a further embodiment of the present invention, the antibacterial bioactive fraction thus obtained has antibacterial activity against gram positive and gram negative bacterial in the range of 200-500 ppm.

Accordingly, a further embodiment of the present invention provides a process for the preparation of antibacterial fraction, which comprises,
i) Powdering the fruits of Cinnamomum zeylanicum to get a particle size 60-80 mesh.
ii) extracting of the above said material with hexane in a Soxhlet extractor at a temperature of 55-60° C. for a period of 6-8 h.
iii) filtering the above extract using Whatman filter paper no. 1 to obtain the particle free extract.
iv) distilling the above extract to recover/recycle the solvent up to 90%.
v) concentrating the above particle free extract at a temperature of 55-60° C.
vi) drying the above concentrated extract using vacuum oven at 40-50° C. under vacuum at 10-25 mm of mercury.
vii) the product thus obtained had antibacterial activity against different Gram positive and Gram negative bacteria in the range of 200-500 ppm.

In an embodiment of the present invention, the yield of hexane extract was found to be 1.5-3.0%.

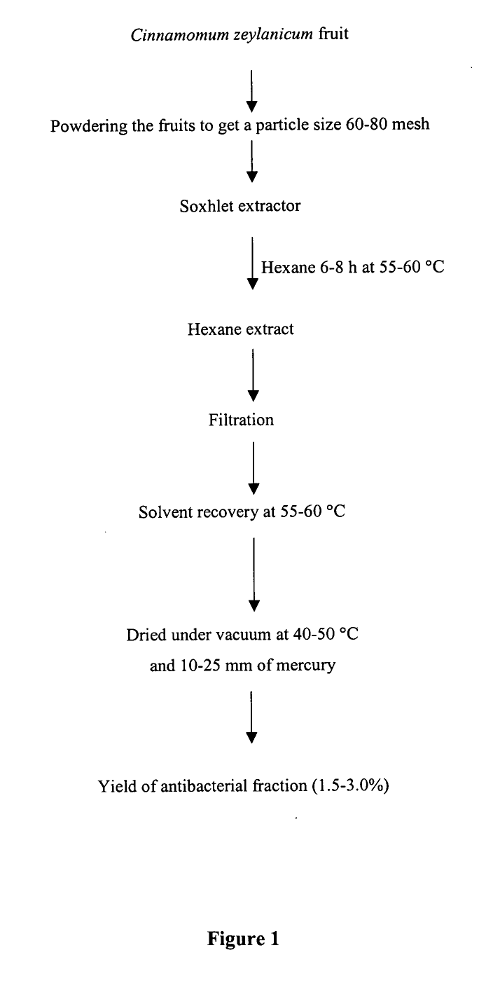

The preparation of antibacterial fraction from the unconventional parts of Cinnamomum zeylanicum was done according to the flow diagram shown in FIG. 1. The novelty of the process includes:
1. This is the first report of preparation of antibacterial fraction from the unconventional parts of Cinnamomum zeylanicum.
2. The invention includes a process to obtain the bioactive fraction from the unconventional parts of Cinnamomum zeylanicum and the fraction so obtained.

The following examples are given by way of illustration of the present invention and therefore should not be constructed to limit the scope of the present invention.

### EXAMPLE 1

50 g fruits of Cinnamomum zeylanicum were powdered using mixer grinder to get a 60 mesh size. The powder was extracted using 200 ml of hexane at 60° C. for 8 h in a Soxhlet extractor. The hexane extract was filtered using Whatman filter paper No. 1 and it was concentrated to recover the 150 ml of solvent. The concentrate was dried in a vacuum oven at 40° C. under 10 mm of vacuum. The yield of extract was 1.4 g.

The antibacterial assay for the extract of Cinnamomum zeylanicum was tested by pour plate method against Bacillus cereus by the method of Negi et al. (J. Agricultural and Food Chemistry 47, 4297-4300, 1999). To flasks containing 20 ml melted nutrient agar, different concentration of test material in propylene glycol were added. Equivalent amounts of propylene glycol were used as controls. One hundred μl (about 103 cfu/ml) of culture was inoculated into the flasks under aseptic conditions. The media was then poured into sterilized petri plates in quadruplet and incubated at 37° C. for 20-24 h for growth. The minimum inhibitory concentration (MIC) was reported as the lowest concentration of the compound capable of inhibiting the complete growth of the bacterium being tested. The MIC value of Cinnamomum zeylanicum fruit extract against Bacillus cereus was 250 ppm.

### EXAMPLE-2

The dried fruits (100 g) of Cinnamomum zeylanicum were powdered in a mixer grinder to get 80 mesh size. The powder was extracted with 400 ml of hexane by using Soxhlet extractor at 55° C. for 8 h. The extract was filtered using Whatman filter paper No 1. and concentrated under vacuum to recover the 360 ml of solvent. The concentrate dried at a temperature of 35° C. and under a reduced pressure at 25 mm of mercury. The yield of hexane extract was 3.0 g.

The antibacterial assay for the extract of Cinnamomum zeylanicum was done by known method. The MIC value of Cinnamomum zeylanicum fruit extract against Bacillus subtilis was 300 ppm.

### EXAMPLE-3

The dried fruits (150 g) of Cinnamomum zeylanicum were powdered in a mixer grinder to get 80 mesh size. The powder was extracted with 600 ml of hexane by using Soxhlet extractor at 55° C. for 8 h. The extract was filtered using Whatman filter paper No 1 and concentrated under vacuum to recover the 520 ml of solvent. The concentrate dried at a temperature of 35° C. and under a reduced pressure at 25 mm of mercury. The yield of hexane extract was 4.7 g.

The antibacterial assay for the extract of Cinnamomum zeylanicum was done by known method. The MIC value of Cinnamomum zeylanicum extract against Bacillus coagulans was 300 ppm.

### EXAMPLE 4

The antibacterial assay for the extract of Cinnamomum zeylanicum was done by known method. The MIC value of Cinnamomum zeylanicum extract against Pseudomonas aeruginosa was 200 ppm.

### EXAMPLE 5

The antibacterial assay for the extract of Cinnamomum zeylanicum was done by known method. The MIC value of Cinnamomum zeylanicum extract against Staphylococcus aureus was 500 ppm.

The advantages of the process are:
1. The process is simple and the solvents used in this process can be regenerated for further use.
2. The raw material has no commercial value at present.

We claim:

1. A composition comprising a bioactive fraction obtained from fruits of Cinnamomum zeylanicum having

| | |
|---|---|
| Moisture: | 4-6% |
| Color: | Greenish white |
| Flavor: | Mild salty flavor |

optionally along with one or more pharmaceutically acceptable additives.

2. A composition as claimed in claim 1, wherein the bioactive fraction is a hexane extract obtained from the fruits of Cinnamomum zeylanicum.

3. A composition as claimed in claim 1, wherein the composition has antibacterial activity against gram positive and gram negative bacterial in the range of 200-500 ppm.

4. A composition as claimed in claim 1, wherein the composition has antibacterial activity against Bacillus cereus, Bacillus subtilis, Bacillus coagulans, Pseudomonas aeruginosa, Staphylococcus aureus.

5. Use of a bioactive fraction obtained from fruits of Cinnamomum zeylanicum having

| | |
|---|---|
| Moisture: | 4-6% |
| Color: | Greenish white |
| Flavor: | Mild salty flavor |

as an antibacterial agent.

6. Use as claimed in claim 5, wherein the bioactive fraction is a hexane extract obtained from the fruits of Cinnamomum zeylanicum.

7. Use as claimed in claim 5, wherein the bioactive fraction has antibacterial activity against gram positive and gram negative bacterial in the range of 200-500 ppm.

8. Use as claimed in claim 5, wherein the bioactive has antibacterial activity against Bacillus cereus, Bacillus subtilis, Bacillus coagulans, Pseudomonas aeruginosa, Staphylococcus aureus.

9. A process for preparing antibacterial bioactive fraction having

| | |
|---|---|
| Moisture: | 4-6% |
| Color: | Greenish white |
| Flavor: | Mild salty flavor |

from the unconventional parts of Cinnamomum zeylanicum, said process comprising the steps of:
(a) extracting the powdered fruits of Cinnamomum zeylanicum with an organic solvent at a temperature in the range of 55-60° C. for a time period in the range of 60-80 mesh.
(b) filtering and concentrating the solvent obtained in step (a) to obtain a concentrate and to recover up to 90% of the solvent;
(c) drying the concentrate obtained in step (b) in a vacuum oven at 40-50° C. under vacuum at 10-25 mm of mercury to obtain the antibacterial bioactive fraction.

10. A process as claimed in claim 9 wherein the organic solvent used is hexane.

11. A process as claimed in claim 10 wherein the yield of hexane extract is about 1.5 to 3.0%.

12. A process as claimed in claim 9 wherein the filtration is carried out by conventional methods.

13. A process as claimed in claim 9 wherein the concentration temperature is of 55-60° C.

14. A process as claimed in claim 9 wherein the antibacterial bioactive fraction thus obtained has antibacterial activity against gram positive and gram negative bacterial in the range of 200-500 ppm.
